(19) United States
(12) Patent Application Publication
(10) Pub. No.: US 2022/0409307 A1
(43) Pub. Date: Dec. 29, 2022

(54) SYSTEMS AND METHODS FOR DETECTING SKIVING IN SURGICAL INSTRUMENTS

(71) Applicant: DePuy Synthes Products, Inc., Raynham, MA (US)

(72) Inventors: Robert Brik, Brookline, MA (US); William J. Fraiser, New Bedford, MA (US)

(21) Appl. No.: 17/825,187

(22) Filed: May 26, 2022

Related U.S. Application Data
(60) Provisional application No. 63/215,115, filed on Jun. 25, 2021.

(51) Int. Cl. A61B 34/30

(52) U.S. Cl. CPC A61B 34/30; A61B 2034/2072

(57) ABSTRACT

Systems, methods, and devices are disclosed for surgical instruments, systems, and methods for detecting skiving of a surgical instrument, such as an instrument used during a robotic or robot-assisted surgery. The embodiments disclosed herein may include one or more sensors adjacent to, coupled to, disposed on, or embedded into an instrument in order to measure deflection thereof during use that may indicate skiving of the instrument. A variety of sensors may be utilized, including strain gauges, resistance-based sensors, fiber optic cables, laser distance measurement units, ultrasonic distance measurement units, optical cable measurement units, etc. In some embodiments, multiple such sensors may be included in an instrument in order to measure magnitude and/or direction of deflection.

### CROSS-REFERENCE TO RELATED APPLICATION

This application claims priority to US Provisional Ser. No. 63/215,115, filed Jun. 25, 2021, the disclosure of which is incorporated herein in its entirety.

### BACKGROUND

In a robotic or robot-assisted surgical procedure, a surgical robotic arm may guide or control one or more surgical instruments, such as a drill, screw, etc. Robot-assisted surgery may provide increased precision and accuracy with regard to the placement of surgical instruments during a procedure and the ability to precisely follow a predetermined surgical plan (e.g., a planned trajectory). For example, spinal surgeries often require precision drilling and placement of screws or other implants in bone or hard tissue. Improper drilling or maneuvering of the spinal body during spinal surgery may be undesirable due to the proximity of the spinal cord and arteries. Further, accurate placement of screws and other implants may be important for a successful outcome.

Robotic surgical systems are capable of very accurate tracking of a position of the surgical instruments used in the system, often achieved using a variety of surgical navigation tracking systems. However, such navigation tracking systems have a limit to their resolution or ability to track movements, especially those movements that are very line or small in magnitude.

One such undesirable movement that may be very difficult to track is skiving of an instrument tip during operation. For example, skiving may occur during the initiation of a drilling operation when a drill tip is set at an entry point in an angled fashion, such that the tip may deflect, “walk,” or otherwise move itself away from the intended entry point before drilling into the hard tissue. Risk of skiving may be increased when, as in many surgical procedures, drilling is performed at predetermined desired trajectories on curved anatomical surfaces, such as certain features of bone.

Accordingly, there is a need for improved surgical devices, systems, and methods that may address these and other shortcomings of prior solutions for drilling into bone or other hard tissue with precision.

### SUMMARY

Systems, methods, and devices are disclosed for surgical instruments, systems, and methods for detecting skiving of a surgical instrument, such as an instrument used during a robotic or robot-assisted surgery. The embodiments disclosed herein may include one or more sensors adjacent to, coupled to, disposed on, or embedded into an instrument in order to measure deflection thereof during use that may indicate skiving of the instrument. A variety of sensors may be utilized, including strain gauges, resistance-based sensors, fiber optic cables, laser distance measurement units, ultrasonic distance measurement units, optical cable measurement units, etc. In some embodiments, multiple such sensors may be included in an instrument in order to measure magnitude and/or direction of deflection.

In some embodiments, a robotic surgical system is provided, comprising a robotic arm, a surgical instrument operably attached to the robotic arm, wherein the surgical instrument comprises: an elongate body extending between a proximal end and a distal end, the distal end being configured to retain an implement to penetratingly contact hard tissue; a channel formed in at least a portion of the elongate body along a longitudinal axis of the elongate body, and a sensor at least partially disposed in the channel, and a controller configured to receive data from the sensor and use the data to determine whether deflection of the elongate body is occurring during use of the surgical instrument.

In some embodiments, a robotic surgical system is provided, comprising a robotic arm extending between a base and an end effector, an instrument mount attached to the end effector and retaining a guide, a surgical instrument rotatably disposed in the guide, the surgical instrument comprising an elongate body extending between a proximal end and a distal end, the distal end being configured to retain an implement to penetratingly contact hard tissue, a sensor, associated with either the guide or the surgical instrument, and a controller configured to receive data from the sensor and use the data to determine whether deflection of the elongate body is occurring during use of the surgical instrument.

In some embodiments, a surgical method is provided, comprising surgical method, comprising providing a controller for a robotic arm extending between a base and an end effector, an instrument mount attached to the end effector and retaining a guide, and an elongate surgical instrument rotatably disposed in the guide, driving the elongate instrument into the hard tissue, and determining deflection of the elongate instrument using a sensor coupled to the elongate instrument or coupled to the guide, wherein the controller is configured to use the determined deflection to determine if a level of skiving that exceeds a predetermined threshold is occurring. In some examples, when the level of skiving exceeds the predetermined threshold, the method further comprises at least one of displaying an alert or ceasing actuation of the elongate instrument.

Further details of these various embodiments are described in more detail below.

### BRIEF DESCRIPTION OF THE DRAWINGS

FIG. 1A is a schematic of a robotic surgical system including a surgical robot with an attached end effector for holding an elongate instrument;

FIG. 1B is a schematic of a robotic surgical system featuring another embodiment of a surgical robot having a movable base cart;

FIGS. 2A-2D are illustrations of example patient anatomy;

FIGS. 3A-3C show end effectors for coupling to a robotic arm;

FIG. 4 shows various elongate instruments and guides,

FIG. 5 shows a distal-end perspective view of an elongate instrument having a sensor for detecting deflection;

FIG. 6 shows a side out-away view of the elongate instrument of FIG. 5:

FIG. 7 shows a detail view of the proximal end of FIG. 6;

FIG. 8 shows a proximal-end perspective view of another embodiment of an elongate instrument having a plurality of sensors for detecting deflection;

FIG. 9A shows a detail view of the proximal end of the elongate instrument of FIG. 8;

FIG. 9B shows a cut-away view of the detail view of FIG. 9A;

FIG. 10 shows a detail cut-away view of the proximal end of yet another embodiment of an elongate instrument having a plurality of sensors for detecting deflection; and

FIG. 11 shows a sectional detail view of a guide-based sensor system for detecting deflection of an elongate instrument disposed within the guide.

### DETAILED DESCRIPTION

The present disclosure provides surgical instruments, systems, and methods for detecting skiving of a surgical instrument, such as during a robotic or robot-assisted surgery. The embodiments disclosed herein may detect, and, in some cases, measure, deflection of an elongate instrument, for example, elongate instruments that are used to penetrate into hard tissues. Furthermore, in some cases, the embodiments may be configured to determine a direction of the deflection. Examples of such instruments include a drill, tap, screw, driver, or other surgical instruments that exert pressure with respect to tissue, such as hard tissue like bone, etc., for example, by being drilled or otherwise driven into tissue.

Detection of deflection in an elongate instrument as it is driven into tissue may indicate the presence of skiving. Skiving is undesirable and may result in a deviation from a target trajectory (e.g., deviation from a planned axis and/or a deviation from an intended (e.g., planned) entry point) of the instrument into the tissue. Measurement of deflection may be beneficial to quantify an amount of skiving. In the event a sufficient level of deflection is detected, the systems and methods disclosed herein may alert a user and/or cease operation of the instrument (e.g., automatically) to prevent further deviation from the target trajectory. Measurement of direction of deflection may be beneficial to quantify the implications of skiving. For example, skiving in one direction may be clinically acceptable, whereas skiving in the opposite direction may have an undesirable effect on the outcome of the procedure.

The embodiments disclosed herein may make use of a number of different sensors adjacent to, coupled to, disposed on, or embedded into an elongate instrument. Examples may include various strain gauges, resistance-based sensors, fiber optic cables, laser distance measurement units, ultrasonic distance measurement units, optical cable measurement units, and others. In certain embodiments, such sensors may be configured to detect magnitude and/or direction of deflection using, in some embodiments, multiple sensors to provide directional sensitivity.

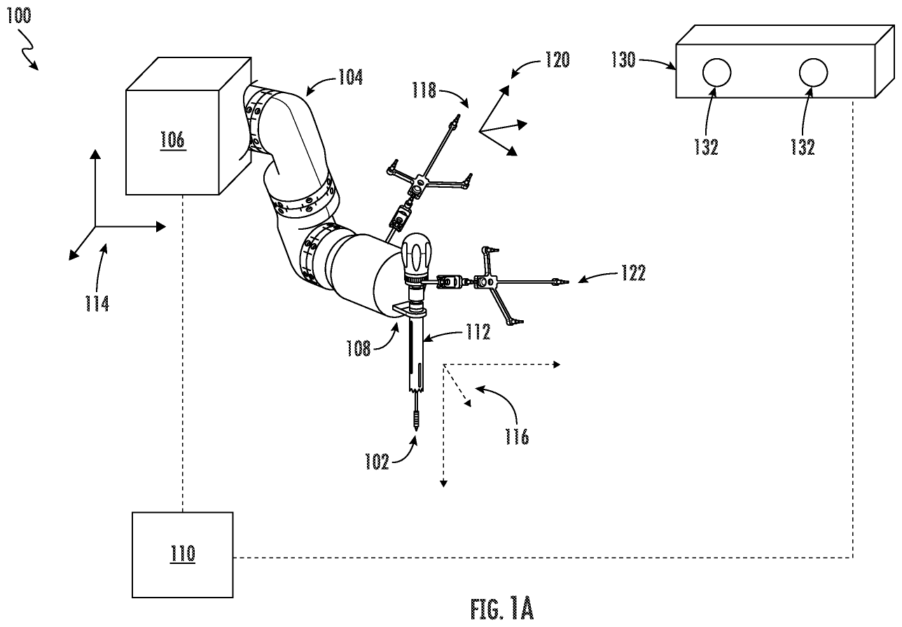

FIG. 1A illustrates a robotic (e.g., robot-assisted) surgical system 100 that may be utilized with the systems and methods described herein to detect skiving of an elongate instrument 102 (which may also be referred to as a surgical tool). The system 100 comprises a robotic arm 104 extending from a base 106, and terminating in an end effector 108 for attaching to the instrument 102. The robotic arm 104 has a plurality of arm segments connected by rotatable joints, the movement of which may be controller by a control system, referred to herein as a controller 110.

The controller 110 may be used to actuate the robotic arm 104 (e.g., control the actuation of each joint) to control movement and thus position the end effector 108, which effects a trajectory of the elongate instrument 102. The controller 110 typically includes power supply, AC/DC converters, motion controllers to power the motors of the actuation units in each joint, fuses, real-time control system interface circuits, and other components conventionally included in surgical robot devices. The controller 110 is configured to receive sensor data from one or more sensors adjacent to, coupled to, disposed on, or embedded into the elongate instrument 102 and to determine deflection of the instrument during use thereof (e.g., that may indicate skiving of the instrument). The controller 110 may also be configured to send an alert (such as of detected skiving). The controller 110 may also be configured to stop (e.g., stop rotation of) the elongate instrument 102. The controller 110 may also be configured to send a notification that instrument use has been ceased due to detected skiving (e.g., via a closed loop command from the controller).

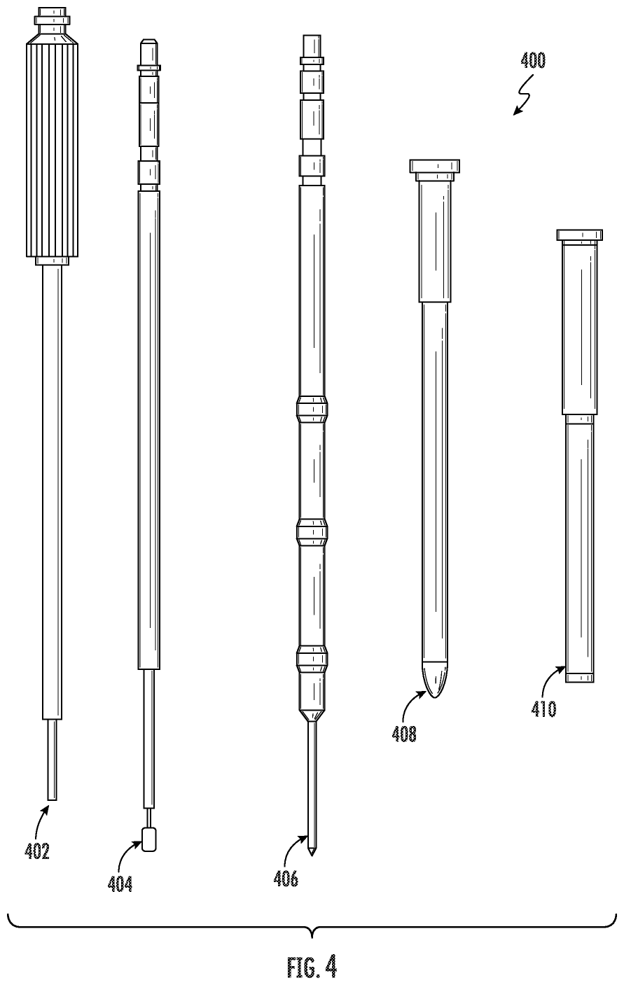

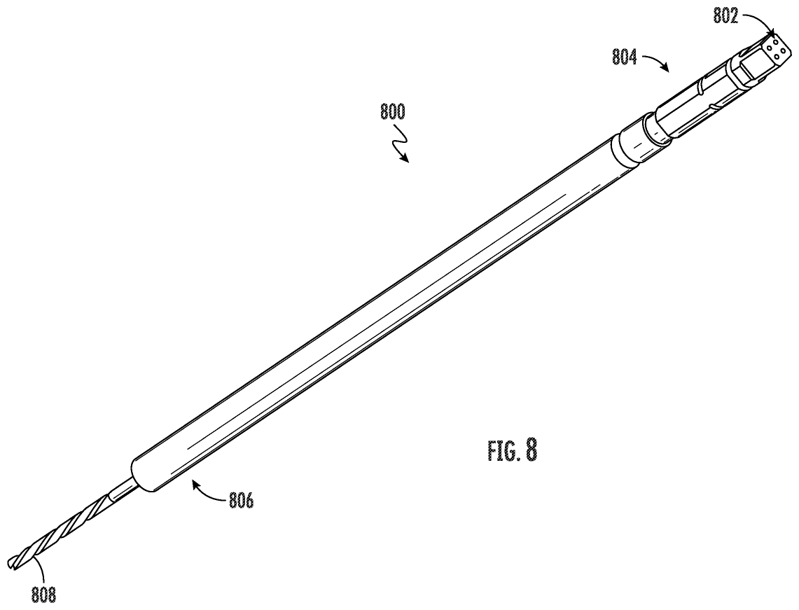

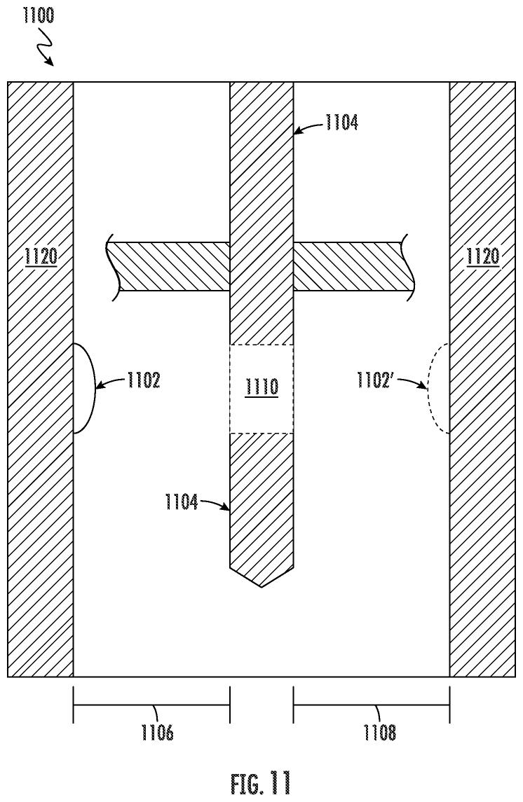

The end effector 108 may comprise an instrument mount or guide 112 configured to receive the instrument 102. Examples of instruments include, for example: a drill bit, saw blade, burr, reamer, mill, knife, or any other implement that could cut or deform bone or other tissue (e.g., penetrate) and is appropriate for use in a given operation (e.g., a drill may be more appropriate in one operation while a rotary burr may be more appropriate in another operation, etc.), and is associated with a sensor as will be described. In some embodiments, the instrument 102 is a sensor-equipped instrument (such as in FIGS. 5, 8, & 10). In some embodiments, the guide 112 is a sensor-equipped guide (such as in FIG. 11). Further, the present disclosure is also contemplated to include use of such instruments by surgical robots, by users with some degree of robotic assistance, and in some cases perhaps without involvement of surgical robots or robotic assistance (e.g., but with a controller configured to determine, based on the sensor data, deflection, deflection and direction of deflection, etc.).

While the illustrated embodiments and accompanying description may refer to a specific surgery, the systems and methods described herein may be utilized in various applications involving robotic, robot-assisted, and non-robotic operations where computer-assisted instrument location are desired and precise adjustment of instrument position may be appropriate. Example applications include knee surgery, such as total knee arthroplasty (TKA), spinal fusion surgery, and other orthopedic surgeries. The teachings of the present disclosure may be applied to such procedures, however, the systems and methods described herein are not limited to these applications.

The robot-assisted surgical system 100 may have a plurality of navigational features to determine a position and orientation in absolute space (with respect to all degrees of freedom of a three-dimension coordinate system), thereby determining a trajectory of the instrument 102. For example, the robotic device as a whole can be said to have a global coordinate system 114, which may be defined in different ways, but generally uses the location of the base 106. A position of various components may be determined, for example, calculated by receiving a position signal from an encoder in each joint of the robotic arm 104. For example, the end effector 108 may be constrained to move about such that the summation of the positions of the joints defines the location of an end effector coordinate system 116 in the global coordinate system 114.

Position may (e.g., may also) be directly measured. A navigation array 118 may be mounted to a distal portion of the robotic arm 104. The navigation array 118 may include one or more markers. In some instances, a measured coordinate system 120 of the array 118 may be used as the global coordinate system 114. A navigation array 122 (additionally or alternatively to the array 118) may be mounted to the end effector 108. The navigation array 118 may include one or more markers. The end effector coordinate system 116 may be defined in different ways, but may refer to the position and orientation of the end effector 108 with respect to the operation of the instrument 102. The array 122 may identify a positioning of the instrument 102 (e.g., received within the guide). In this manner, the array 122 may help provide complete positioning information (e.g., of the instrument 102) to a user (e.g., a surgical robot system, surgeons, etc.).

A tracking unit 130, such that the relative pose or three-dimensional position and orientation of the array 118 and/or 122, as well as any other navigation arrays present in an operating theater, e.g., such as an array coupled to patient anatomy (not shown), a surgical table, etc. (not shown), may be tracked in real time and shared to the controller 110 and any additional planning system. In some embodiments, the tracking unit 130 may include one or more navigation system cameras 132 that may capture a location of the one or more markers in the arrays 118 and/or 122. The tracking unit 130 may measure the relative motions between any and all coordinate systems in real time. Real time can, in some embodiments, mean high frequencies greater than twenty Hertz, in some embodiments in the range of one hundred to five hundred Hertz, with low latency, in some embodiments less than five milliseconds. The location information captured from the markers of an array may thus identify a location of the component to which it is coupled in three-dimensional space given the known and precise relationship between the array and the component. For example, the array 122 may be configured to identify the depth position of the instrument 102, such as a tip, without being permanently connected or fastened to the instrument.

In a manner similar to the arrays discussed above, a further array (not shown) may be coupled with a patient or other structure in the operating environment (e.g., a surgical table, etc.), to assist, with keeping tracking of an anatomy of interest, for example, a pedicle of the spine. A patient coordinate system may be defined in different ways (e.g., using an array coupled to the patient), but may refer to the position and orientation of the patient with respect to the end effector 108 or instrument 102. The tracking system 130 may track these objects for purposes of displaying their relative positions and orientations to the surgeon and, in some cases, for purposes of controlling and/or constraining manual manipulation of the instrument relative to virtual boundaries associated with the patient's anatomy.

FIG. 1B illustrates another embodiment of a system 100′ that may be used with a surgical robot device 100′. The surgical system of FIG. 1B may be similar to the surgical system of FIG. 1A, in that it may include a robotic arm 104 having multiple arm segments joined together by a plurality of joints and a sensor-equipped instrument 102. The robotic arm 104 may be coupled to a movable cart at its base 106. Additionally, one or more navigation arrays 140 may be coupled to various parts of the robot device 100. Only representative array 140 is shown, but a plurality of arrays and a navigation system may be employed as discussed above in connection with FIG. 1A. An external device 150 may communicate with the controller (not depicted). The device 150 may be a display, a computing device, remote server, etc., configured to allow a surgeon or other user to input data directly into the controller. Such data may include patient information and/or surgical procedure information. The device 150 may display information from the controller, such as alerts or notification that instrument use has been ceased (e.g., via a closed loop command from the controller). Communication between the device 150 and the controller may be wireless (e.g., near-field communication (NFC), Wi-Fi, Bluetooth, Bluetooth LE, ZigBee, and the like) or wired (e.g., USB or Ethernet).

In summary, the trajectory of an instrument, such as in a static state or only moving along a single axis, may be determined with great precision. However, there are cases where movements off a planned trajectory are too fine or small in magnitude (e.g., at least initially) for a surgical navigation tracking system to detect, but that are nonetheless undesirable because they may result in, for example, pedicle breach or compromised screw purchase in the context of spine surgery. For example, spinal fusion is often augmented by stabilizing the vertebrae with fixation devices, such as metallic screws, rods, and plates, to facilitate bone fusion. In spinal fusion, as well as other surgeries, the accuracy with which the screws and other implants are placed in the bone may have a direct effect on the outcome of the procedure. Moreover, risk of skiving may be increased when, as in many surgical procedures, drilling is performed at predetermined desired trajectories on curved anatomical surfaces, such as certain features of bone.

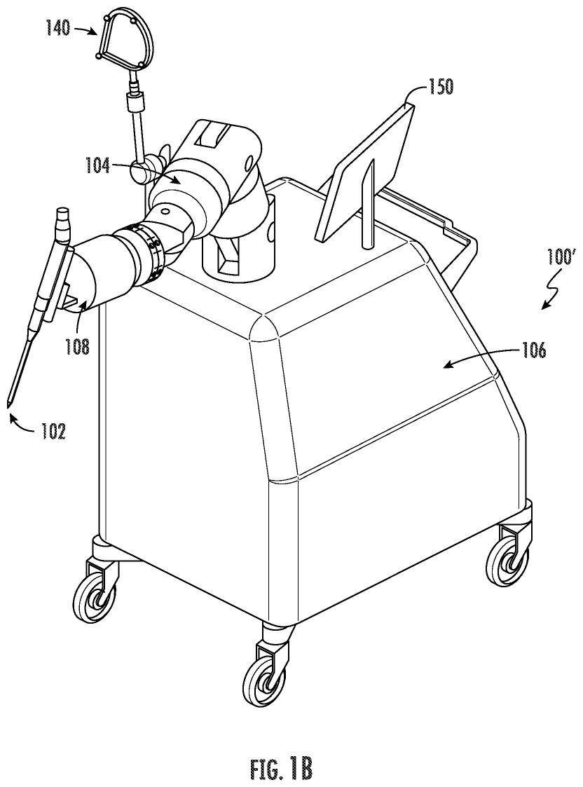

FIGS. 2A-2D are illustrations of a patient's spinal column 251. Specifically, FIG. 2A shows a rear perspective view of the spinal column 251 having discs 252 and a spinal cord 254. FIG. 2B shows the same view without the discs 252 and the spinal cord 254. The spinal column 251 has a plurality of hard bone vertebrae 256, each of which has a body 258 and a vertebral arch. The vertebral arch is formed by two pedicles 260 extending posteriorly from the main body 258 and leading to two laminae 262 that terminate medially at a spinous process 266 and laterally at two transverse processes 267.

FIGS. 2C and 2D show a side view and a rear view, respectively, of the spinal column 251 during a surgical procedure such as a spinal fusion. Specifically, the surgical procedure shows a pedicle screw-rod system 270 including a rod 272 coupled with rod connectors 274 and a bone anchor or screw 276. Due to the intricacies of working in proximity to the spinal column 251, spinal surgery procedures may require great precision and accuracy to avoid undesirable outcomes. For example, such procedures typically require spinal fixation assemblies to be delivered directly (i.e., substantially perpendicular to the midline of the patient's spinal column) into a lateral mass or pedicle 260 of a target vertebra 256. In light of this trajectory, slight deviations from a desired delivery trajectory may result in penetration of a distal portion of the assembly (e.g., a pointed tip of a bone screw shank) into the spinal canal (which contains the spinal cord 254) or the foramina 268 (see FIG. 2A) of the exiting nerve root, which may be undesirable. As a further disadvantage, the limited bone mass and/or bone density that may be found in the lateral mass portion of a vertebra 256 may limit the area available for contacting the fixation assembly 270, thereby hindering the ability to effectively position the fixation assembly 270 within the vertebra 256.

For the reasons described above, it may be important that penetrating (e.g., awling, drilling, tapping, driving into, and/or other cutting) of hard tissue (e.g., bone) be planned carefully. Such surgical pre-planning may include a target trajectory for drilling a hole into bone, said trajectory including target entry point, orientation, diameter, and depth. Spinal geometry is variable, however, and often not flat at the optimal entry point (e.g., pedicle surface) for the drill on the target trajectory. When a pointed cylindrical drill contacts a curved surface (or a flat surface at such an angle that relative curvature is created between the components), the drill may have a tendency to move off target trajectory or a predetermined entry point as drilling is initiated (e.g., skiving). Skiving results in a screw trajectory through the bone that is different than the planned target trajectory, and may produce undesirable results, such as pedicle breach or compromised screw purchase.

A navigation or tracking array (such as array 118, array 122, array 140, etc.) may conceptually detect misalignment of an elongate instrument during awing, drilling, tapping, driving, or other cutting operations, however, such arrays may have resolutions or minimum trackable movement sizes that are too large to detect initial movements associated with skiving. In some cases, detection by an array of a surgical instrument being off track may not be possible until significant deviation has occurred, making correction more difficult.

Accordingly, sensor-equipped instruments, systems, and methods, as will be described in more detail may detect skiving more quickly and precisely by using sensors that measure lateral forces indicative of deflection of the elongate instrument. This may be accomplished using a variety of sensors that may be adjacent to, coupled to, disposed on, or embedded into the elongate instrument (e.g., a drill, tap, screw, driver, or other tool) and in communication with a surgical system, such as the controller 110 (FIG. 1A). As a result, any deflection of the elongate instrument may be measured during use, equated to skiving, and, should skiving (e.g., sufficiently-high levels of deflection) be detected, a user may be alerted and/or tool (e.g., instrument) use may be ceased (e.g., activation of the instrument may be automatically stopped) to allow for correction of any deviation from a target trajectory.

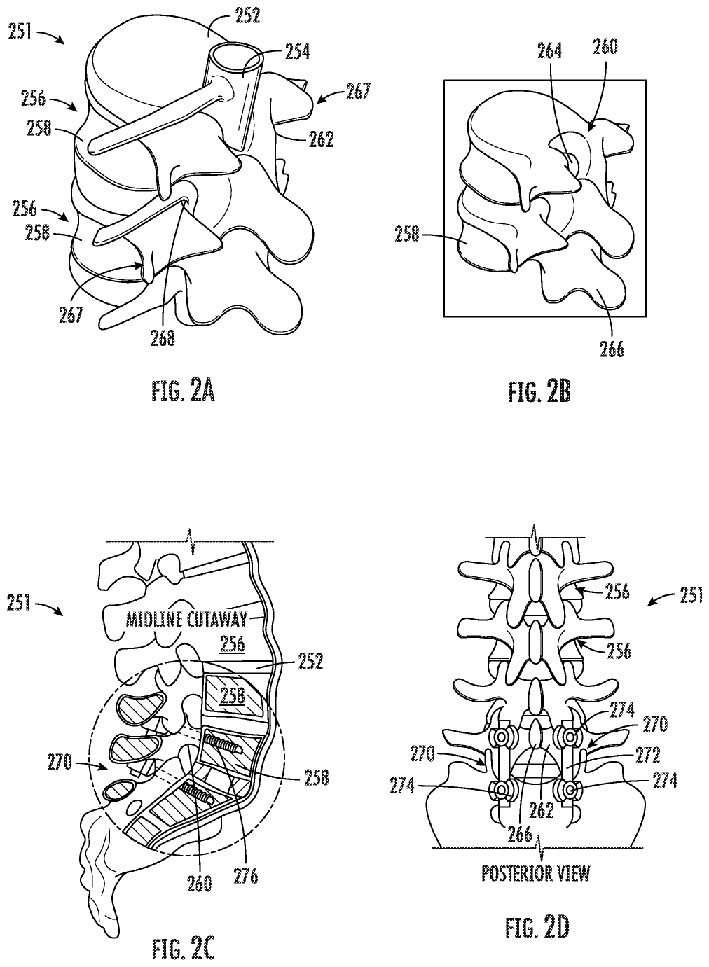

FIGS. 3A-3C illustrate various embodiments of end effectors, instrument mounts, and guides that may be used with surgical instruments in the surgical robotic systems disclosed herein.

FIG. 3A shows an end effector 302 including an instrument mount 304. The instrument mount 304 may receive a guide 306 (e.g., an access port or instrument guide) at a distal end thereof.

FIG. 3B shows a drill-tap-screw (DTS) guide end effector 308. An instrument mount 310 may hold the DTS guide or sleeve 312 and receive an elongate instrument 314, e.g., a drill, tap, screw, or combination instrument, with a perpendicular trajectory relative to a longitudinal axis of the end effector.

FIG. 3C shows another embodiment of a DTS guide end effector 316 that includes an instrument mount 318 that may hold a guide 320. An elongate instrument 322 is held in the guide 320 at an angled or oblique trajectory relative to a longitudinal axis of the end effector.

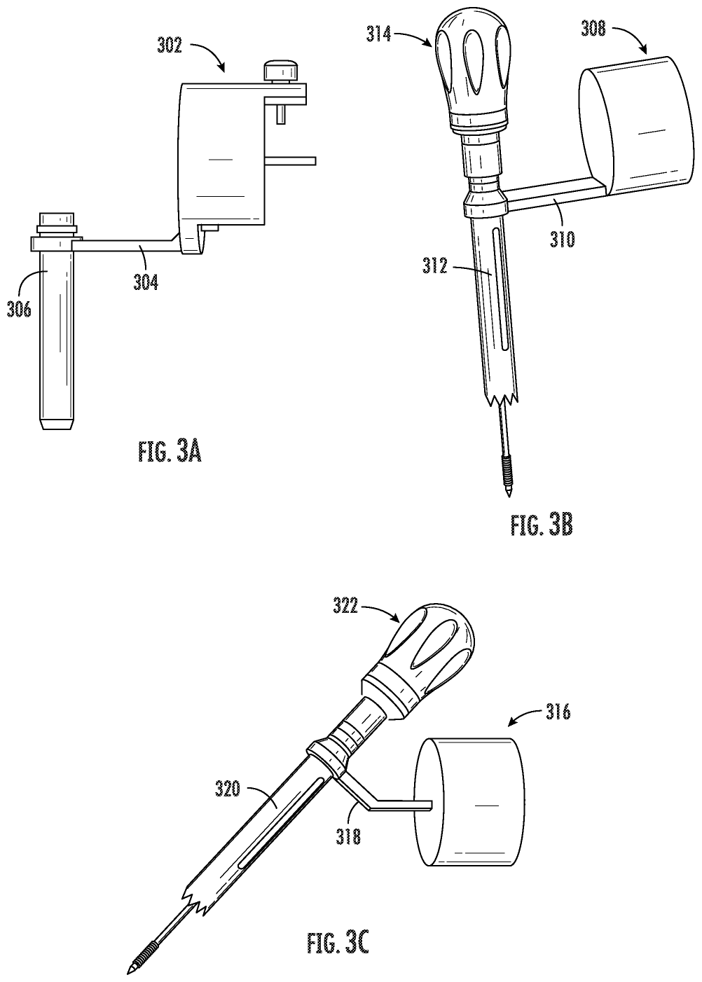

FIG. 4 illustrates a set 400 comprising other examples of elongate instruments which include various drilling, tapping, and/or driving instruments 402, 404, 406 (or alternatively, awling instruments (not depicted)). FIG. 4 also shows associated guides 408, 410 that may be coupled to a robotic arm (such as robotic arm 104) and utilized to guide delivery of the instruments 402, 404, or 406 to patient anatomy. Any of a variety of instruments and associated guides may be interchangeably coupled to a robotic arm (such as via an end effector) in place of those depicted.

FIGS. 5-10 show embodiments of elongate instruments with embedded sensors for detecting skiving of the instrument.

FIG. 5 illustrates an embodiment of sensor-equipped surgical tool or instrument 500. The instrument has a proximal portion or end 502, an elongate body 504, and distal portion or end 506. The proximal end 502 may be configured to interface with an actuator that drives a drill bit 510 disposed at the distal end 506, e.g., in a rotary motion. For example, the proximal end 502 may include flats 508 or other features to allow torque transmission during driving. The body 504 may have varying lengths according to the configuration of the surgical system in which the instrument 500 is utilized. The drill bit 510 of the distal end 506 is configured to drill into hard tissue, such as bone.

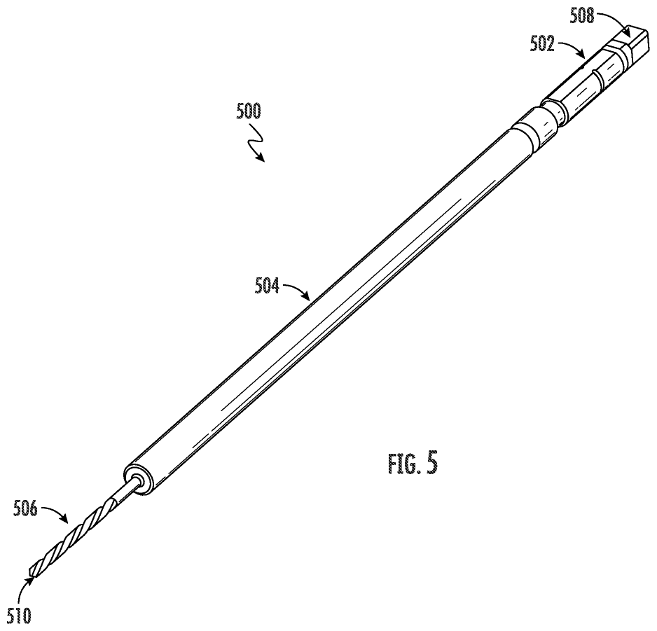

FIG. 6 illustrates a cut-away view of the instrument 500 showing a sensor 602 embedded within a channel 604 formed in the body 504 (FIG. 5). As depicted, the channel 604 extends along an entire longitudinal length of the body 504 of the instrument 500. Alternatively, the channel 604 may extend for only a portion of the entire longitudinal length of the body 504, such as 60% or greater, 70% or greater, 80% or greater, less than 100%, less than 90%, etc. The channel 604 may be formed by drilling or machining. The channel 604 is aligned with a longitudinal axis defined by the body 504. In an embodiment, the channel 604 is concentric to the longitudinal axis of the body 504. In an alternative embodiment, the channel 604 is off-axis relative to the longitudinal axis of the body 504.

The sensor 602 may have a variety of different forms. For example, the sensor 602 may be a strain gauge, a fiber optic cable, a resistance-based sensor such as a conductive wire, conductive rubber, conductive fiber, conductive fabric, or liquid metal microchannel, etc. The sensor 602 may be embedded inside the body 504 of the instrument, as shown in the figure, or may run along an outer surface of the instrument in some embodiments. The sensor 602 may detect a change in the channel 604, such as, for example, a change in conductivity, which may indicate deflection of the channel. A deflection of the channel 604 indicates deflection of the instrument 500. A deflection of the instrument 500 indicates that skiving is occurring. For example, the controller 110 (FIG. 1A) may receive output (e.g., data) from the sensor 602 and determine that deflection of the instrument 500 is not occurring or is occurring. If deflection of the instrument 500 is occurring, the controller may determine that skiving is occurring, and alert a user and/or cease operation of the instrument (for example, via a closed loop command from the controller) to prevent further deviation from the target trajectory.

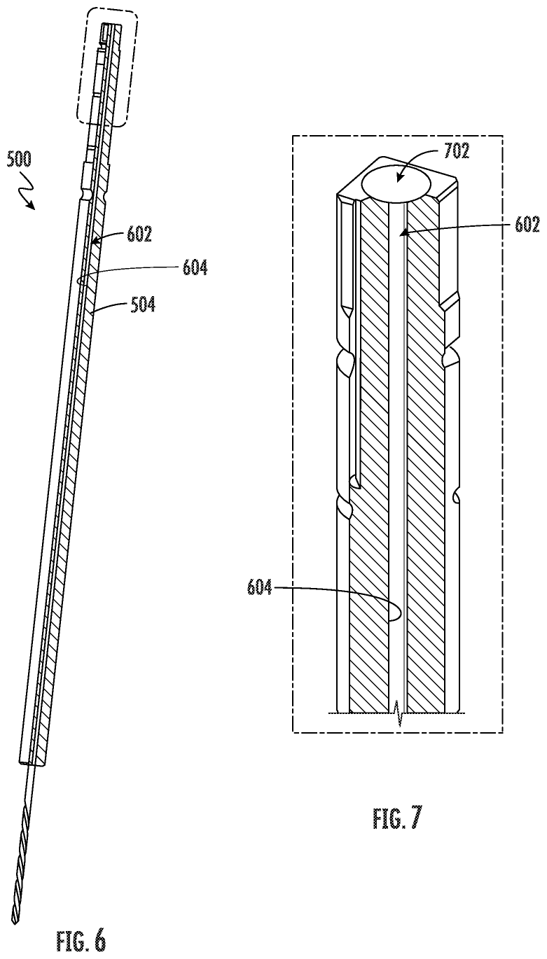

FIG. 7 illustrates a detail view of the proximal end 502 showing a connector 702 for coupling to the sensor 602 and reading or measuring its output during use. A number of different connectors 702 may be utilized to couple the sensor 602 to a controller, such as controller 110 in FIG. 1A. For example, a simple contact as illustrated in FIG. 7 may be utilized. In other embodiments where a rotating interface is required between the elongate instrument 500 and another component, a slip ring connection may be utilized to allow free rotation of the instrument during awling, drilling, tapping, driving, or other cutting, etc., while maintaining a connection to the sensor 602.

Certain examples of sensors 602 may provide information on magnitude of deflection, but no information regarding directionality of the deflection, which may be useful if, for example, to compensate by correcting the trajectory of the elongate instrument 500 (for example, by a controller determining to correct the trajectory). Accordingly, it may be desirable in some embodiments to include a sensor or sensors capable of providing information on both magnitude and direction of deflection. One such option that may be utilized in a single-sensor embodiment is a fiber optic cable as the sensor 602. In particular, the data provided by light traveling through a fiber optic cable and reflecting back may be more robust than strain gauge output, for example, and may be utilized to determine not only magnitude of deflection but also direction of the deflection. One or more fiber Bragg gratings (FBG) may be utilized to reflect or transmit particular wavelengths of light, and placement of one or more gratings in the channel 604 may be utilized to determine a direction of deflection in addition to magnitude.

Alternatively, multiple sensors may be adjacent to, coupled to, disposed on, or embedded into an elongate instrument to provide information on direction of deflection in addition to magnitude. For example, inclusion of two sensors may provide directional information in one plane, and inclusion of three sensors may give multi-axis direction information.

FIG. 8 shows another embodiment of an elongate instrument 800 having multiple sensors 802 embedded therein to provide information on instrument deflection magnitude and direction. The elongate instrument 800 is similar to the instrument 500 mentioned above, so detailed description of its construction is omitted. As can be appreciated, this embodiment has a plurality of channels that run parallel to a longitudinal axis defined by the elongate instrument 800, but are spaced apart. As best seen in FIG. 98B, these channels receive the sensors 802. The four sensors 802 may each be selected from a strain gauge, a fiber optic cable, a resistance-based sensor such as a conductive wire, conductive rubber, conductive fiber, conductive fabric, or liquid metal microchannel, etc. Preferably, all four sensors 802 are of the same type. In this embodiment, differences between outputs of the sensors may be used (e.g., by the controller 110 FIG. 1A) similar to the output of the sensor 602 described above. In this embodiment, it is likely that a slip ring or other kind of multi-channel rotational coupling be utilized to provide communication with each sensor 802 during use.

The instrument 800 has a proximal portion or end 804 and a distal portion or end 806. The proximal end 804 may be configured to interface with an actuator that drives a drill bit 808 disposed at the distal end 806 to drill into hard tissue, such as bone, e.g., in a rotary motion (e.g., the proximal end 804 may include features to allow torque transmission during driving). The instrument 800 may have varying lengths according to the configuration of the surgical system in which the instrument 500 is utilized.

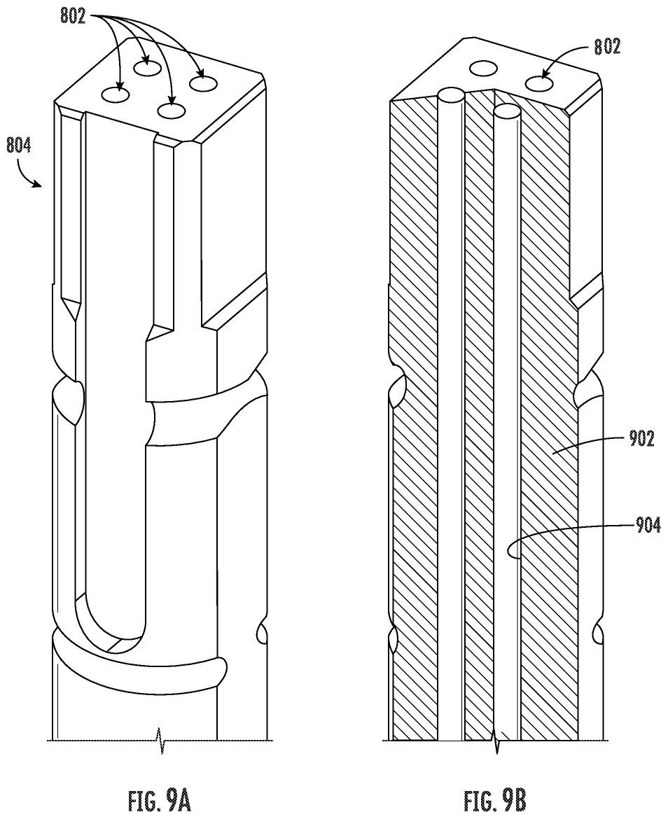

FIG. 9A shows a detail perspective view of a proximal portion 804 of the instrument 800 and the four sensors 802 embedded therein. FIG. 9B shows a cutaway view of FIG. 9A. A plurality of channels 904 may be formed in the body 902, such as by drilling or machining. Each channel 904 may extend for all of, or only a portion of, the entire longitudinal length of the body 902, such as 60% or greater, 70% or greater, 80% or greater, 100%, less than 100%, less than 90%, etc.

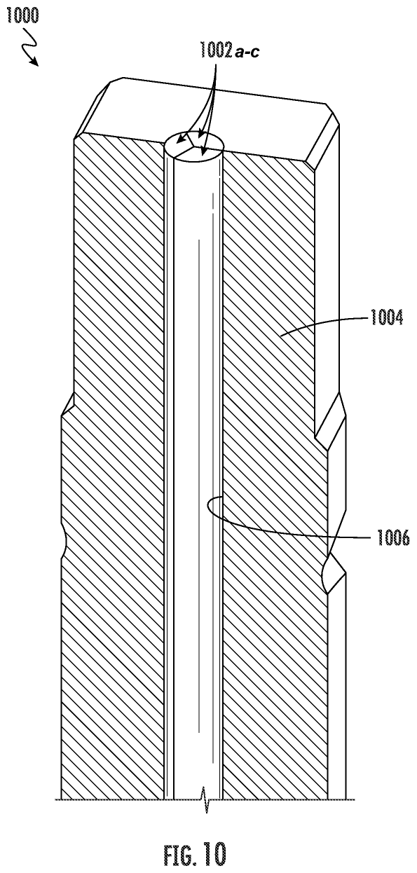

FIG. 10 illustrates a proximal cut-away view of yet another embodiment of an elongate instrument 1000. The elongate instrument 1000 is a single-channel, multi-sensor-equipped elongate instrument. A plurality of distinct sensors 1002a-c are formed into a single cylindrical assembly that is embedded in a channel 1006 of the body 1004 and running along a length thereof. The three sensors 1002a-c may be selected from a strain gauge, a fiber optic cable, a resistance-based sensor such as a conductive wire, conductive rubber, conductive fiber, conductive fabric, or liquid metal microchannel, etc., provided that no sensors are the same. In the illustrated embodiment, three sensors 1002a-c are shown but different numbers of sensors are possible, e.g., two, four, or more. Optionally, a rotational coupler capable of delivering individual data from each sensor 1002a-c may be utilized to connect a controller with the instrument 1000.

The channel 1006 is aligned with a longitudinal axis defined by the body 1004. In an embodiment, the channel 1006 is concentric to the longitudinal axis of the body 1004. In an alternative embodiment, the channel 1006 is off-axis relative to the longitudinal axis of the body 1004. The channel 1006 may extend for all of, or only a portion of, the entire longitudinal length of the body 1004, such as 60% or greater, 70% or greater, 80% or greater, 100%, less than 100%, less than 90%, etc.

Sensors of the present disclosure (e.g., sensors 602, 802, 1002a-c) may be configured such that signals received by a controller from the sensors may be decoupled, which may lead to a determination of deflection magnitude and direction of the elongate instrument, as well as determination of movement mode in more complicated cases (e.g., where a particular surgical instrument encounters a torsion in addition to lateral deflection, etc.). Decoupling sensor signals may be beneficial as the surgical instrument may move relative to the cannula or guide in different modes. By decoupling the signal(s) received from the sensors coupled to, disposed on, or embedded in the surgical instrument, a controller of the present disclosure may identify each movement of the instrument and provide information on the magnitude and direction of deflection of the instrument from a target trajectory.

In some embodiments, a statistical model may be programmed into the controller to read and decouple the signals received from the sensor(s). In other embodiments, analytical decoupling may be performed. By way of example, an analytical decoupling of data from sensors may be analytically akin to a strain gauge rosette. In an embodiment, each of the four sensors 802 may be a resistance-based sensor or strain sensor. The signal transmissions from each sensor may be read and analyzed to identify the instrument movement by magnitude and direction of strain and shear. For example, the controller may identify a magnitude and direction of deflection by analyzing components of the data transmitted by each sensor.

In instances or applications in which a signal from one or more sensors cannot be easily decoupled, higher techniques of signal processing may be deployed to capture and identify precise movement of the instrument. For example, multimodal machine learning techniques may be applied to enhance movement detection and measurement. For example, the controller may include a machine learning module that may build a framework through which data from the sensors may be interpreted such that movement mode(s) and respective magnitude(s) and direction(s) may be determined. The machine learning module may receive as input pre-programed information pertaining to instrument movement, such as calibration test results on the instrument or a similar instrument. The machine learning module may receive as further inputs data from various other sources, such as data related to the procedure being performed, data from other sensors coupled to the robot, etc. The machine learning module may take these inputs and construct models of instrument movement. The controller may then apply the machine learning framework to signals received from the sensors to understand and capture precise movement based on the sensor data.

Still another embodiment for determining both magnitude and direction of deflection in an elongate instrument may include a hybrid approach utilizing software. For example, in an embodiment an elongate instrument with a single embedded sensor may be utilized, such as the instrument 500 described above. Data from this sensor 602 that provides information about magnitude of deflection only, may be combined with knowledge of the procedure being performed via software in a controller to determine the direction of deflection that is likely to be experienced. More particularly, if the controller has positioned the instrument relative to bone or other tissue in a manner that provides a distinct angle. e.g., due to the trajectory of the instrument entering bone, the curvature of the bone at the point of insertion, etc., it may be possible to determine with a reasonably high degree of accuracy that, should skiving occur, it would occur in a particular direction dictated by the relative arrangement of the instrument and anatomy being drilled into, tapped, driven into, or otherwise cut, etc.

In still other embodiments, sensors may be incorporated into other components of a surgical system (e.g., such as the guide) to provide information on the bending, deflection, or skiving of an elongate instrument.

FIG. 11 depicts a sectional view of a guide-based sensor system 1100 for detecting deflection of an elongate instrument 1104 (such as a drill, tap, driver, or other tool) disposed within a guide 1120 (such as the guide 306 shown in FIG. 3A). In one such embodiment, for example, one or more sensors 1102 may be provided on an inner surface of the guide 1120. The sensor 1102 may be configured to measure a distance 1106 from the guide 1120 (or sensor 1102) to the elongate instrument 1104. Since the instrument 1104 may ideally be positioned exactly in the center of the guide 1120 and may have a known diameter, the distances 1106, 1108 from the instrument outer surface to the inner surface of the guide should remain constant and even.

In some embodiments, only one sensor 1102 is used and therefore only a variation in the distance 1106 is determined (e.g., by an associated controller (such as the controller 110 of FIG. 1A) to determine deflection of the instrument 1104). Any change in the measured distance from the inner wall of the guide 1120 to the instrument 1104 may indicate deflection or bending of the instrument. Moreover, a positive or negative change in the distance 1106 may also indicate a general direction of deflection (e.g., away from or toward the sensor 1102). In some embodiments, the sensor 1102 may be a laser distance measurement unit, an ultrasonic distance measurement unit, an optical cable measurement unit, etc. The distance 1108 may be calculated, or alternatively, a second senor 1102′ may be provided on another inner surface of the guide 1120, to determine the distance 1108.

In certain embodiments, the instrument 1104 may be provided with a flat portion 1110 on an outer surface thereof that may be configured to reflect light, sound, etc., back to the sensor 1102 during each rotation of the elongate instrument. In addition, in some embodiments more than one sensor, such as sensor 1102 and sensor 1102′ may be utilized to increase data refresh rates and accuracy. In such embodiments, the sensors 1102, 1102′ may be arranged around the circumference of the guide 1120 in a variety of manners. For example, two sensors may be arranged on opposite sides of the guide 1120's inner surface, or may be offset ninety degrees from one another to capture possible deflection in two planes. In still other embodiments, larger numbers of sensors like sensor 1102 (e.g., three, four, or more) may be arranged around the circumference of the inner surface of the guide sleeve 1120 in a variety of manners (e.g., both even and uneven spacing, etc.).

In a first embodiment, a robotic surgical system is provided, comprising a robotic arm, a surgical instrument operably attached to the robotic arm, wherein the surgical instrument comprises, an elongate body extending between a proximal end and a distal end, the distal end being configured to retain an implement to penetratingly contact hard tissue; a channel formed in at least a portion of the elongate body along a longitudinal axis of the elongate body, and a sensor at least partially disposed in the channel, and a controller configured to receive data from the sensor and use the data to determine whether deflection of the elongate body is occurring during use of the surgical instrument. In some examples, the controller is further configured to use the data to determine a magnitude of deflection of the elongate body. In some examples, the controller is further configured to use the data to determine a direction of deflection of the elongate body. In some examples, the controller is further configured to use the data to determine a magnitude and direction of deflection of the elongate body.

In some examples, the sensor is at least one of a strain gauge, a fiber optic cable, or a resistance-based element that changes electrical resistance with bending. In some examples, the sensor is a fiber optic cable and further includes a plurality of gratings configured to communicate any of magnitude or direction of deflection of the elongate body. In some examples, the sensor is a resistance-based element. In some examples, the resistance-based element is at least one of a conductive wire, a conductive rubber, a conductive fiber, a conductive fabric, or a liquid metal microchannel. In some examples, the channel includes at least three sensors to communicate a direction of deflection of the elongate body. In some examples, the surgical instrument further comprises a plurality of channels, each channel having an associated sensor to communicate a direction of deflection of the elongate body. In some examples, the controller is further configured to use the deflection data to determine if a level of skiving that exceeds a predetermined threshold is occurring. In some examples, if the controller determines a level of skiving that exceeds a predetermined threshold is occurring, the controller is further configured to display an alert and/or the controller is further configured to cease actuation of the surgical instrument.

In a second embodiment, a robotic surgical system is provided, comprising a robotic arm extending between a base and an end effector, an instrument mount attached to the end effector and retaining a guide, a surgical instrument rotatably disposed in the guide, the surgical instrument comprising an elongate body extending between a proximal end and a distal end, the distal end being configured to retain an implement to penetratingly contact hard tissue, a sensor, associated with either the guide or the surgical instrument, and a controller configured to receive data from the sensor and use the data to determine whether deflection of the elongate body is occurring during use of the surgical instrument. In some examples, the sensor is disposed in the guide, for example, the sensor is one or more laser distance measuring units, ultrasonic distance measuring units, or optical cable measuring units, or the sensor is configured to measure a radial distance between the elongate instrument and an inner wall of the guide. In some examples, the controller is further configured to use the data to determine at least one of a magnitude of deflection of the elongate body or a direction of deflection of the elongate body. In some examples, the sensor is disposed in a channel in the elongate instrument, and the sensor is at least one of a strain gauge, a fiber optic cable, or a resistance-based element that changes electrical resistance with bending.

In a third embodiment, a surgical method is provided, comprising surgical method, comprising providing a controller for a robotic arm extending between a base and an end effector, an instrument mount attached to the end effector and retaining a guide, and an elongate surgical instrument rotatably disposed in the guide, driving the elongate instrument into the hard tissue, and determining deflection of the elongate instrument using a sensor coupled to the elongate instrument or coupled to the guide, wherein the controller is configured to use the determined deflection to determine if a level of skiving that exceeds a predetermined threshold is occurring. In some examples, when the level of skiving exceeds the predetermined threshold, the method further comprises at least one of displaying an alert or ceasing actuation of the elongate instrument.

In a fourth embodiment, a surgical instrument is provided, comprising an elongate body extending between a proximal end and a distal end, the distal end being configured to retain an implement to penetratingly contact hard tissue, a channel formed in the elongate body along a longitudinal axis of the elongate body, and a sensor disposed in the channel, wherein the sensor is configured to output data that may be used to detect deflection of the elongate body during use. The sensor may be at least one of a strain gauge, a fiber optic cable, or a resistance-based element that changes electrical resistance with bending. When the sensor includes a fiber optic cable, the fiber optic cable may further include a plurality of gratings configured to communicate any of magnitude or direction of deflection of the elongate body. When the sensor includes the resistance-based element, the resistance-based element may be a conductive wire, a conductive rubber, a conductive fiber, a conductive fabric, or a liquid metal microchannel. The sensor may be embedded within the elongate body. The instrument may include a plurality of sensors. The instrument may include at least three sensors configured to communicate any of magnitude or direction of deflection of the elongate body. The instrument may include at least four sensors configured to communicate any of magnitude or direction of deflection of the elongate body. A proximal portion of the elongate body may have a diameter greater than that of the distal end to house the sensor.

In a fifth embodiment, a surgical system is provided, comprising a surgical instrument according to any of the foregoing examples and a guide surrounding the instrument. The surgical system may further comprise a controller for receiving signals from the sensor and determining if deflection of the surgical instrument has occurred. The instrument may be configured to rotate relative to the guide. The surgical system may further comprise a slip ring in contact with the sensor. The surgical system may further comprise an end effector for holding the guide. The surgical system may further comprise a robotic arm for holding the end effector.

In a sixth embodiment, a surgical system is provided, comprising a surgical system, comprising an elongate instrument having a longitudinal axis extending from a proximal end to a distal end thereof, a distal end portion of the elongate body being configured to interface with hard tissue, a guide surrounding the elongate instrument and attached to a robotic arm, and a sensor attached to the guide and configured to detect deflection of the elongate body instrument during use. The sensor may be disposed in the guide and configured to measure a position of the elongate instrument relative to the guide. The sensor may measure a radial distance between the elongate instrument and an inner wall of the guide from a plurality of positions arranged around an inner circumference of the guide. The sensor may include a laser distance measurement unit, an ultrasonic distance measurement unit, or an optical cable measurement unit. The instrument may be configured to rotate relative to the guide. The surgical system may further comprise a controller for receiving signals from the sensor and determining if deflection of the surgical instrument has occurred.

In a seventh embodiment, a surgical method is provided, comprising inserting an elongate instrument through a guide to access hard tissue, driving the elongate instrument into the hard tissue, and measuring deflection of the elongate instrument using a sensor coupled to the elongate instrument or coupled to a guide concentric to the elongate instrument. The surgical method may further comprise alerting a user upon detection of deflection of the elongate instrument above a threshold level. The surgical method may further comprise ceasing to drive the elongate instrument into the hard tissue upon detection of deflection of the elongate instrument above a threshold level. The surgical method may further comprise alerting a user that actuation of the elongate instrument has been ceased.

1. A robotic surgical system, comprises:
a robotic arm;
a surgical instrument operably attached to the robotic arm, wherein the surgical instrument comprises:
an elongate body extending between a proximal end and a distal end, the distal end being configured to retain an implement to penetratingly contact hard tissue;
a channel formed in at least a portion of the elongate body along a longitudinal axis of the elongate body; and
a sensor at least partially disposed in the channel; and
a controller configured to receive data from the sensor and use the data to determine whether deflection of the elongate body is occurring during use of the surgical instrument.

2. The robotic surgical system of claim 1, wherein the controller is further configured to use the data to determine a magnitude of deflection of the elongate body.

3. The robotic surgical system of claim 2, wherein the controller is further configured to use the data to determine a direction of deflection of the elongate body.

4. The robotic surgical system of claim 1, wherein the sensor is at least one of a strain gauge, a fiber optic cable, or a resistance-based element that changes electrical resistance with bending.

5. The robotic surgical system of claim 1, wherein the sensor is a fiber optic cable and further includes a plurality of gratings configured to communicate any of magnitude or direction of deflection of the elongate body.

6. The robotic surgical system of claim 1, wherein the sensor is a resistance-based element.

7. The robotic surgical system of claim 6, wherein the resistance-based element is at least one of a conductive wire, a conductive rubber, a conductive fiber, a conductive fabric, or a liquid metal microchannel.

8. The robotic surgical system of claim 1, wherein the channel includes at least three sensors to communicate a direction of deflection of the elongate body.

9. The robotic surgical system of claim 1, wherein the surgical instrument further comprises a plurality of channels, each channel having an associated sensor to communicate a direction of deflection of the elongate body.

10. The robotic surgical system of claim 1, wherein the controller is further configured to use the deflection data to determine if a level of skiving that exceeds a predetermined threshold is occurring.

11. The robotic surgical system of claim 10, wherein, if the controller determines a level of skiving that exceeds a predetermined threshold is occurring, the controller is further configured to display an alert.

12. The robotic surgical system of claim 10, wherein, if the controller determines a level of skiving that exceeds a predetermined threshold is occurring, the controller is further configured to cease actuation of the surgical instrument.

13. A robotic surgical system, comprises:
a robotic arm extending between a base and an end effector;
an instrument mount attached to the end effector and retaining a guide;
a surgical instrument rotatably disposed in the guide, the surgical instrument comprising an elongate body extending between a proximal end and a distal end, the distal end being configured to retain an implement to penetratingly contact hard tissue;
a sensor, associated with either the guide or the surgical instrument; and
a controller configured to receive data from the sensor and use the data to determine whether deflection of the elongate body is occurring during use of the surgical instrument.

14. The robotic surgical system of claim 13, wherein the sensor is disposed in the guide.

15. The robotic surgical system of claim 14, wherein the sensor is one or more laser distance measuring units, ultrasonic distance measuring units, or optical cable measuring units.

16. The robotic surgical system of claim 13, wherein the sensor is configured to measure a radial distance between the elongate instrument and an inner wall of the guide.

17. The robotic surgical system of claim 16, wherein the controller is further configured to use the data to determine at least one of a magnitude of deflection of the elongate body or a direction of deflection of the elongate body.

18. The robotic surgical system of claim 13, wherein the sensor is disposed in a channel in the elongate instrument, and the sensor is at least one of a strain gauge, a fiber optic cable, or a resistance-based element that changes electrical resistance with bending.

19. A surgical method, comprising:
providing a controller for a robotic arm extending between a base and an end effector, an instrument mount attached to the end effector and retaining a guide, and an elongate surgical instrument rotatably disposed in the guide;
driving the elongate instrument into the hard tissue; and
determining deflection of the elongate instrument using a sensor coupled to the elongate instrument or coupled to the guide, wherein the controller is configured to use the determined deflection to determine if a level of skiving that exceeds a predetermined threshold is occurring.

20. The surgical method of claim 19, wherein, when the level of skiving exceeds the predetermined threshold, the method further comprises at least one of:
displaying an alert; or
ceasing actuation of the elongate instrument.
